(19) United States
(12) Patent Application Publication
(10) Pub. No.: US 2024/0404620 A1
(43) Pub. Date: Dec. 5, 2024

(54) FRAMEWORK FOR PROTEIN DESIGN FROM PARTIAL SEQUENCE WITH MEMORY-EFFICIENT GLOBAL ATTENTION METHOD

(71) Applicant: The Chinese University of Hong Kong, Hong Kong (CN)

(72) Inventors: Pheng Ann HENG, Hong Kong (CN); Guangyong CHEN, Hong Kong (CN); Xinyi ZHOU, Hong Kong (CN)

(21) Appl. No.: 18/326,154

(22) Filed: May 31, 2023

(51) Int. Cl. G16B 15/20; G16B 15/30; G16B 40/20

(52) U.S. Cl. CPC G16B 15/20; G16B 15/30; G16B 40/20

(57) ABSTRACT

A machine-learning based method and systems for protein sequence design are provided. The method includes generating a portion of a sequence and removing noise in input residue context, encoding and processing the portion of the sequence and backbone structure to obtain graph features, performing memory-efficient global graph attention layers to propagate the graph features and learn global residue interactions; and generating an entire sequence non-iteratively. The performing memory-efficient global graph attention layers includes enabling each residue node to learn residue interactions and gather information from the entire sequence while maintaining memory efficiency. The edge features of the memory-efficient global graph attention layers include interatomic distances and direction vectors.

### REFERENCE TO SEQUENCE LISTING

The Sequence Listing for this application is labeled “CUHK208.xml” which was created on May 11, 2023 and is 10,958 bytes. The entire content of the sequence listing is incorporated herein by reference in its entirety.

### BACKGROUND OF THE INVENTION

Computational Protein Design, which is to design proteins with specific structures or functions [1], has been a powerful tool for the exploration of sequence or topology space not yet visited by evolutionary process [2-4] and discovery of proteins with improved properties [5]. It has enabled success in membrane protein design [6] and enzyme design [7]. Inverse Protein Folding (IPF), as one of the sub-tasks of Computational Protein Design, tackles the problem of finding amino acid sequences that can fold into a given three-dimensional (3D) backbone structure [8] and is of great importance for hosting a particular function often presupposes acquiring a specific backbone structure.

The ways to model and utilize residue interactions have been the focuses of various IPF algorithm development. In traditional methods, energy functions are designed to approximate backbone-sequence compatibility. Residue-pair interaction modeling is usually derived from databases by leveraging statistical preferences for particular residue pairs in a simplified local environment to estimate inter-residue energies [5, 9, 10]. However, the increasing computational complexity limits the statistical estimation of multi-residue interactions that are conditional on a more fine-grained representation of the local environment. [10, 11].

In recent years, deep learning has been widely and successfully applied to protein structure modeling and prediction [12, 13], due to its ability to automatically learn complex non-linear many-body interactions from data. Various research projects have been carried out to investigate deep learning assisted IPF [4, 14, 15]. Early methods often model protein structures as sequences of independent residues [16, 17] or atom point clouds [4, 15] and adopt a non-autoregressive decoding scheme as demonstrated in FIG. 1A. Nevertheless, their independence assumption prevents them from learning complex residue inter-dependence and limits their performance. On the other hand, some recent works use proximity graphs to represent protein structures, where residues are nodes and residue interactions are directly modeled as edges. Generally, a masked encoder-decoder architecture with an autoregressive decoding method is used as shown in FIG. 1B [18-21]. Recently, a similar decoding scheme has been proposed in ABACUS-R as shown in FIG. 1C [22]. This method assumes that all neighbor residue types are known when decoding a central residue. Starting from a random initial sequence, it recursively updates residues based on their neighborhood until convergence. However, the dependency on previous predictions has proven to be prone to error accumulation [23, 24]. In particular, noisy residue information is introduced into the context and propagated through the graph structure. Recovering a target residue would be easier and more accurate if more high-quality residue interactions were available and utilized.

### BRIEF SUMMARY OF THE INVENTION

There continues to be a need in the art for improved designs and techniques for methods and systems for a machine-learning assisted approach to meet the challenges of the protein structure modeling and prediction.

According to an embodiment of the subject invention, a machine-learning based method for protein sequence design is provided, comprising receiving information of residues of a protein sequence; determining if any residue of the protein sequence is known; performing entire sequence design if it is determined that no residue of the protein sequence is known; and performing partial sequence design if it is determined that at least one residue of the protein sequence is known. The performing entire sequence design comprises performing an entropy-based prediction-selection method in combination with a base model to remove noise in input residue context. Moreover, the performing an entropy-based prediction-selection method comprises computing an entropy of predicted distributions at each position, retaining residues having entropies lower than or equal to a threshold value, and masking other residues having entropies greater than the threshold value. The base model can be a GVP-GNN model, a ProteinMPNN model, a ProteinMPNN-C model, or an ESM model.

In another embodiment of the subject invention, another machine-learning based method for protein sequence design is provided. The method comprises generating a portion of a sequence and removing noise in input residue context; encoding and processing the portion of the sequence and backbone structure to obtain graph features; performing memory-efficient global graph attention layers to propagate the graph features and learn global residue interactions; and generating entire sequence non-iteratively. The performing memory-efficient global graph attention layers comprises enabling each residue node to learn residue interactions and gather information from the entire sequence while maintaining memory efficiency. Moreover, the performing memory-efficient global graph attention layers comprises constructing a K-nearest neighbor graph from the backbone structure and node features contain dihedral angles, forward and backward vectors. Edge features of the memory-efficient global graph attention layers include interatomic distances and direction vectors. The edge features are encoded by GVP layers to obtain structural embeddings. In each layer of the memory-efficient global graph attention layers, every residue node globally attends to other residues. An attention score is calculated from both the node and the edge features to determine amount of information that a target node gathers from another node. For node pairs that are not directly connected by an edge, a learnable pseudo edge feature is configured for attention calculation. In addition, each layer learns a separate pseudo edge feature that is shared by all non-existing edges. The attention score is then used to weight and sum up the node and the edge features, generating updated node features. The edge features are updated by the updated node features. Further, the generating entire sequence non-iteratively comprises generating the entire sequence from the node features from the last layer non-iteratively.

In certain embodiment of the subject invention, a computer program product is provided, comprising a non-transitory computer-executable storage device having computer readable program instructions embodied thereon that when executed by a computer cause the computer to perform machine-learning based method for protein sequence design, the computer-executable program instruction comprising generating a portion of a sequence and removing noise in input residue context; encoding and processing the portion of the sequence and backbone structure to obtain graph features; performing memory-efficient global graph attention layers to propagate the graph features and learn global residue interactions; and generating entire sequence non-iteratively.

### BRIEF DESCRIPTION OF THE DRAWINGS

FIGS. 1A-1C are schematic representations of different methods of utilizing inter-residue features according to prior art.

FIG. 1D is a schematic representation of a method based on partial input sequence with selected high-quality amino acid information, according to an embodiment of the subject invention.

FIG. 2 is a schematic representation of a model architecture in which a partial sequence, either given or constructed from existing model's generation, and the backbone structure are encoded and processed to obtain the graph features, wherein several memory-efficient global graph attention layers are employed to propagate the graph features and learn global residue interactions, and generate the sequence in one shot, according to an embodiment of the subject invention.

FIG. 3A is a graph plot showing sequence recovery without entropy-based mask drops significantly, according to an embodiment of the subject invention.

FIG. 3B is a confusion matrix of the ESM model, according to an embodiment of the subject invention.

FIG. 3C is a bar graph showing recovery breakdown to hydrophilic and hydrophobic residues, according to an embodiment of the subject invention.

FIG. 3D is a bar graph showing recovery breakdown to residues with different secondary structures, according to an embodiment of the subject invention.

FIG. 3E shows that the sequence designed by the ESM model for 2KCD is predicted to fold into a structure more similar to the native one, according to an embodiment of the subject invention.

FIG. 3F shows the predicted structures of sequences designed for 3A57, according to an embodiment of the subject invention.

FIG. 4A is a bar graph showing recovery breakdown to hydrophilic and hydrophobic residues, according to an embodiment of the subject invention.

FIG. 4B is a bar graph showing recovery breakdown to residues on different secondary structures, according to an embodiment of the subject invention.

FIG. 4C shows Alphafold2 predicted structures of designed sequences for 1Y52 and 1Y2U, with design shells plotted in atoms, wherein sequences designed by the method provides better recovery of the design shell structures, according to an embodiment of the subject invention.

FIG. 5A is a schematic representation of the process of computing the quality score of one target mutation site, according to an embodiment of the subject invention.

FIG. 5B show graphs demonstrating the mutation sites recommended by the ProteinMPNN-C model and by the model of the subject invention, being marked in red and blue, respectively, according to an embodiment of the subject invention.

FIG. 5C shows the improvement of variants recommended by the ProteinMPNN-C model and the model of the subject invention in indel activity relative to TnpB WT, according to an embodiment of the subject invention.

FIG. 6A shows hydrogen bonds between two target residues (HIS 9, ILE 70 on 2KCD) and their most attended residues, according to an embodiment of the subject invention.

FIG. 6B shows a salt bridge and hydrogen bonds between ASP 70 of T4-lysozyme and its most attended residues, according to an embodiment of the subject invention.

FIG. 6C shows a disulfide bond between CYS 99 and CYS 94 of human Ero1-alpha (Q96HE7), according to an embodiment of the subject invention.

FIG. 6D shows model recovery rates with respect to different percentages of residues masked on base model sequences, according to an embodiment of the subject invention.

FIG. 6E shows memory usage of models with and without pseudo edge features, according to an embodiment of the subject invention.

### BRIEF DESCRIPTION OF THE SEQUENCES

SEQ ID NO: 1: EMX1-PCR-seq-F1 primer
SEQ ID NO: 2: EMX1-PCR-seq-F2 primer
SEQ ID NO: 3: EMX1-PCR-seq-F3 primer
SEQ ID NO: 4: EMX1-PCR-seq-F4 primer
SEQ ID NO: 5: EMX1-PCR-seq-F5 primer
SEQ ID NO: 6: EMX1-PCR-seq-R1 primer
SEQ ID NO: 7: EMX1-PCR-seq-R2 primer
SEQ ID NO: 8: EMX1-PCR-seq-R3 primer
SEQ ID NO: 9: EMX1-PCR-seq-R4 primer
SEQ ID NO: 10: EMX1-PCR-seq-R5 primer
SEQ ID NO: 11: EMX1 spacer sequence

### DETAILED DISCLOSURE OF THE INVENTION

Embodiments of the subject invention are directed to a machine-learning based method and system for protein sequence design based on selection of high-quality residue interactions.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not prelude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention pertains. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

When the term “about” is used herein, in conjunction with a numerical value, it is understood that the value can be in a range of 90% of the value to 110% of the value, i.e. the value can be +/−10% of the stated value. For example, “about 1 kg” means from 0.90 kg to 1.1 kg.

As used herein, the term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

In this disclosure, the term “isolated nucleic acid” molecule means a nucleic acid molecule that is separated from other nucleic acid molecules that are usually associated with the isolated nucleic acid molecule. Thus, an “isolated nucleic acid molecule” includes, without limitation, a nucleic acid molecule that is free of nucleotide sequences that naturally flank one or both ends of the nucleic acid in the genome of the organism from which the isolated nucleic acid is derived (e.g., a cDNA or genomic DNA fragment produced by PCR or restriction endonuclease digestion). Such an isolated nucleic acid molecule is generally introduced into a vector (e.g., a cloning vector or an expression vector) for convenience of manipulation or to generate a fusion nucleic acid molecule. In addition, an isolated nucleic acid molecule can include an engineered nucleic acid molecule such as a recombinant or a synthetic nucleic acid molecule. A nucleic acid molecule existing among hundreds to millions of other nucleic acid molecules within, for example, a nucleic acid library (e.g., a cDNA or genomic library) or a gel (e.g., agarose, or polyacrylamide) containing restriction-digested genomic DNA, is not an “isolated nucleic acid”.

In this application, the terms “polypeptide”, “peptide”, and “protein” are used interchangeably herein to refer to a polymer of amino acids. The terms apply to amino acid polymers in which one or more amino acid residues are artificial chemical mimetics of corresponding naturally occurring amino acids, as well as to naturally occurring amino acid polymers and non-naturally occurring amino acid polymers, including those comprising post-translational modifications. As used herein, the terms encompass amino acid chains of any length, including full-length proteins, wherein the amino acid residues are linked by covalent peptide bonds, as well as multi-subunit proteins wherein two or more covalently linked chains of amino acids are associated by covalent bonds or non-covalent interactions.

In describing the invention, it will be understood that a number of techniques and steps are disclosed. Each of these has individual benefits and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

According to the embodiments of the subject invention, the machine-learning based method and system for protein sequence design comprises two design components.

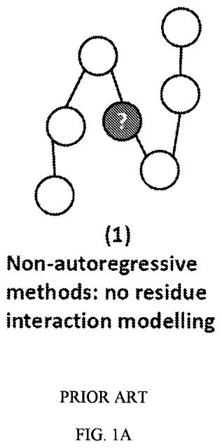

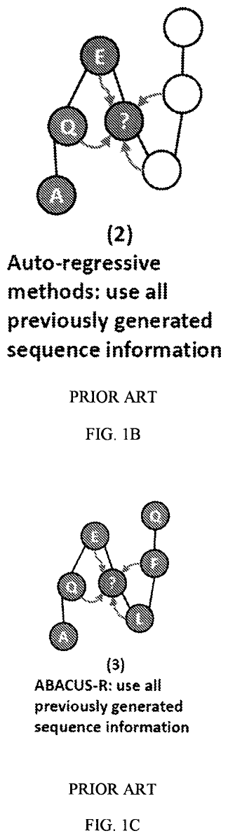

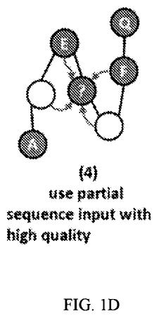

(1) It is assumed that a portion of the sequence is already known and the model takes a partial sequence as input as well as the backbone structure during both training and inference. During the training phase, explicitly introducing ground truth residue information can facilitate the model to learn and exploit residue interactions more effectively, increasing native sequence recovery by up to 8% as shown by the experiment results. During the inference phase, a prediction-selection method is configured to utilize high quality residue information. Referring to FIG. 1D, the method based on partial input sequence with selected high-quality amino acid information is shown.

(2) A memory-efficient global graph attention model is adopted to fully utilize the denoised context, allowing each residue node to learn residue interactions and gather information from the entire sequence while maintaining the memory efficiency.

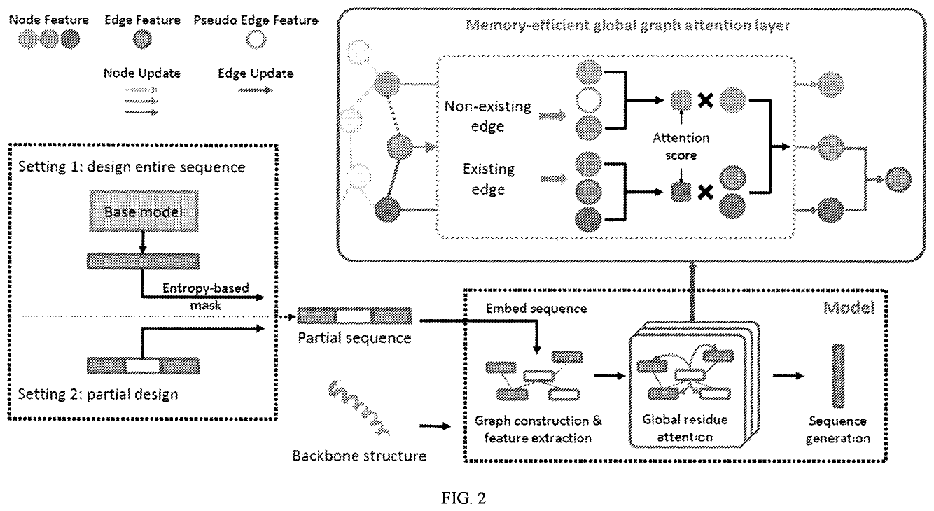

Partial Sequence Input Method: A portion of the sequence is assumed to be known. When training the model, some residues are randomly selected and their types are made visible. During the evaluation, two design settings, which are shown in FIG. 2, are considered.

In one embodiment, when some residues are unknown and need to be designed, the other known residues can serve as an oracle partial input sequence.

In another embodiment, when no residues are known and the entire sequence needs to be designed, an entropy-based prediction-selection method is adopted to utilize high-quality predictions from the existing models. Specifically, given a sequence generated by a conventional model, which is referred to as the base model, the entropy of the predicted distribution at each position is computed. The residues with low entropy, for example, the residues having entropics lower than or equal to a threshold value, are retained and other residues having entropies greater than the threshold value are masked, assuming that models are more confident with low-entropy predictions. Results of experiments demonstrate that precisions of around 99% can be achieved for the residues with the lowest 10% entropy. Thus, the method can effectively remove a significant amount of noise in the input residue environment.

Moreover, different from the conventional methods, the method of subject invention generates the sequence in one shot without iterative design and dependence on previous predictions, resulting in faster sequence design and ensuring higher-quality residue interactions. Accordingly, the model of subject invention generates sequences with high native recovery even with relatively poor base models and achieves state-of-the-art native sequence recovery when better partial sequences are available for input. The strategy for partial input construction is applicable to any existing protein design models, as there are no requirements for the base model architecture.

Memory-efficient Global Graph Attention Method: To learn and leverage residue interactions, the attention method, which has been proven effective in modeling global dependencies for sequential data, is adopted. However, adapting attention to the graph domain can be challenging. Existing works either abandon the original global view [18, 26, 27] or require fully connected graphs to support the global attention calculation [28, 29], which raises the memory complexity from linear to quadratic in node numbers. Further, edge features are not fully leveraged in these existing methods, as they only participate in attention calculations but cannot be updated or used to update node features [18, 26, 28, 30]. Nevertheless, it has been shown that the edge features are crucial for protein structure modeling [20].

To address these issues, according to the embodiments of the subject invention, a model with memory-efficient global graph attention layer is provided as shown in FIG. 2. A K-nearest neighbor graph is constructed from the backbone structure and node features contain dihedral angles, forward and backward vectors. The edge features include interatomic distances and direction vectors. These features are encoded by GVP layers to obtain structural embeddings. To embed the input partial sequence, unknown amino acids are treated as a special type and encoded along with the known amino acids to obtain sequence embeddings. The two types of embeddings, namely, structural and sequence, are concatenated and processed by the global attention layers. In each layer, every residue node globally attends to other residues. An attention score is calculated from both the node and the edge features to determine the amount of information that a target node gathers from another node. For node pairs that are not directly connected by an edge, a learnable pseudo edge feature is configured for attention calculation.

Each layer of the model learns a separate pseudo edge feature that is shared by all non-existing edges. The attention score is then used to weight and sum up the node and the edge features, producing the updated node features. The edge features are also updated by the new node features. Finally, the model generates the sequence from the node features from the last layer in one shot. Therefore, the memory-efficient global graph attention layer allows for global residue attention while eliminating the need for fully connected graph construction by learning the pseudo edge features. The residues are able to leverage the global interactions and the whole-structure features.

The experiment results demonstrate that the method according to the embodiments of the subject invention is effective in handling both entire sequence design and partial sequence design settings. Higher native sequence recovery is achieved for several sequence design benchmarks. In particular, for partial sequence design, the model for the task of single-point mutant design of Transposon-associated transposase B is validated. This is a special case of partial sequence design where only one residue can be modified. The model can generate stable and high-quality sequences and successfully identify six variants with improved gene editing activity out of the 20 mutants recommended by the model.

### Results

The model according to the embodiments of the subject invention is trained on CATH 4.2 training set containing 18,204 structures.

### Entire Sequence Design

A base model is employed when designing the entire sequence. The method according to the embodiments of the subject invention performed in combination with each base models is tested for inverse protein folding.

GVP-GNN is trained on the same training set mentioned above.

ProteinMPNN is trained on selected PDB structures clustered into 25,361 clusters. The same model architecture for training on the same training set is denoted as ProteinMPNN-C and is used for fair comparison.

ESM is trained on CATH 4.3 training set with 16,153 structures and 12 million additional structures predicted by Alphafold2.

Table 1 below provides median sequence recovery rate and nssr scores of the test results of the method according to the embodiments of the subject invention performed in combination with different base models for three benchmarks. The model according to the embodiments of the subject invention can obtain good performance with relatively poor base models and achieve the highest recovery and nssr with better base models.

| | CATH | | TS50 | | Latest PDB | |
|---|---|---|---|---|---|---|
| | Recovery % | nssr % | Recovery % | nssr % | Recovery % | nssr % |
| Conventional | 41.30 | 60.85 | 44.02 | 63.59 | 47.97 | 66.18 |
| GVP-GNN | | | | | | |
| GVP-GNN of | 49.71 | 67.90 | 53.75 | 69.33 | 57.76 | 74.18 |
| Subject Invention | | | | | | |
| Conventional | 42.64 | 60.82 | 45.16 | 60.85 | 49.61 | 66.21 |
| ProteinMPNN | | | | | | |
| ProteinMPNN of | 51.46 | 68.80 | 54.10 | 71.14 | 59.49 | 75.27 |
| Subject Invention | | | | | | |
| Conventional | 44.94 | 63.72 | 48.93 | 67.39 | 55.30 | 71.43 |
| ProteinMPNN-C | | | | | | |
| ProteinMPNN-C | 51.04 | 68.93 | 54.52 | 71.65 | 60.47 | 75.94 |
| of Subject | | | | | | |
| Invention | | | | | | |
| Conventional | 55.25 | 71.48 | 55.78 | 72.02 | 63.19 | 77.31 |
| ESM | | | | | | |
| ESM of Subject | 57.87 | 74.07 | 58.43 | 75.44 | 65.68 | 79.57 |
| Invention | | | | | | |

The experiments are conducted for following three benchmarks.

CATH: CATH 4.2 dataset is a standard dataset for IPF training and evaluation. Its test split of 1,120 structures is evaluated.

TS50: TS50 is a benchmark set of 50 protein chains proposed by [17]. It has been used by a number of previous works [15, 31, 32].

Latest PDB: The latest published structures in PDB are collected as another benchmark to validate the model's ability to generalize for new structures. Protein structures released after Jan. 1, 2022 with a single chain of length less than 500 and resolution 2.5° A are selected, resulting in 1,975 protein structures.

Two metrics on all benchmarks: Sequence recovery and native sequence similarity recovery (nssr) are reported [33]. A pair of residues is considered similar and contributes to the nssr score if their BLOSUM62 score 0. Compared with recovery when only residue identity is considered, nssr takes residue similarity into account and provides a more specific comparison between two sequences.

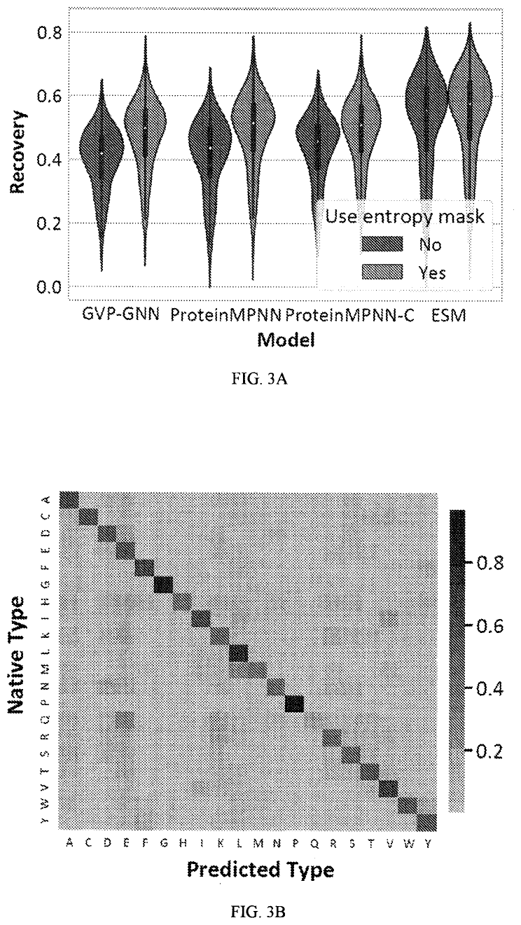

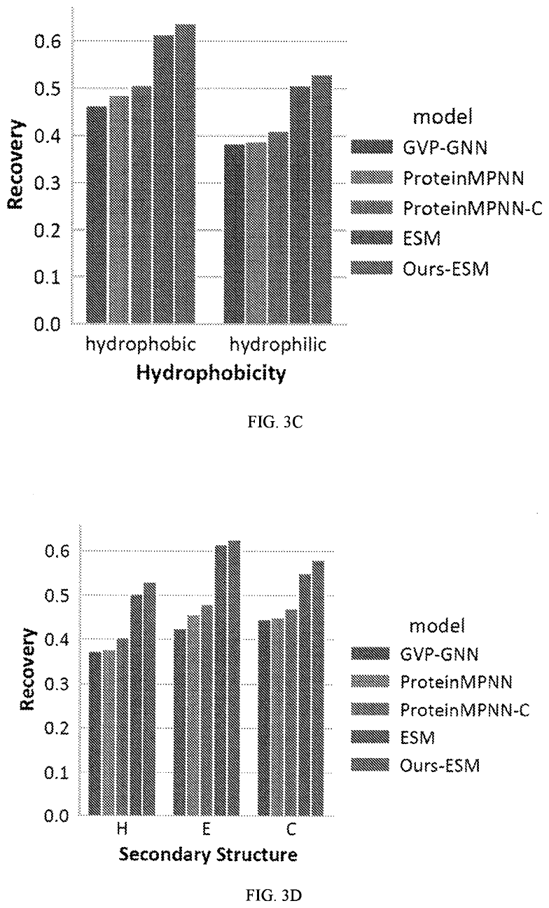

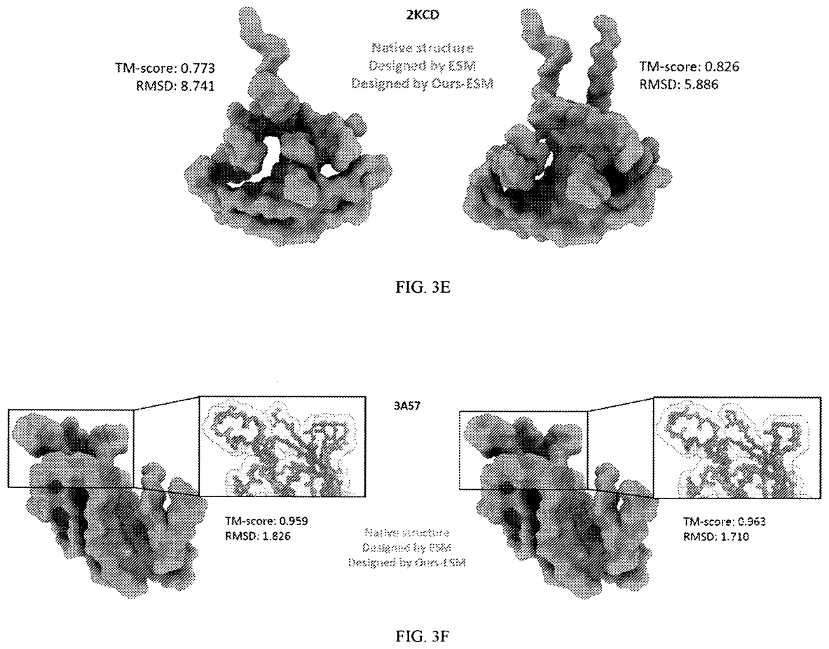

In Table 1 above, the median recovery rate and the nssr scores when using partial sequences from different base models are reported, and the results of the models according to the embodiments of the subject invention and the results of the base models are compared. Among all the base models, the ESM model achieves the best performance, highlighting the effectiveness of data augmentation. The method the method according to the embodiments of the subject invention performed on different base models consistently achieves high recovery rates and nssr scores even with relatively poor base models, demonstrating its ability to denoise input residues and recover native sequences from them. Additionally, when partial sequences with higher quality are available, the method according to the embodiments of the subject invention performed on different base models outperforms the state-of-the-art model that uses 12 million additional structures for augmentation. To validate the effectiveness of entropy-based masking in selecting high-quality residue predictions, the masking operation is removed and the entire predicted sequence is given to the model. Resulted recovery rate on the CATH test set is plotted in FIG. 3A. Removing entropy masks results in a large drop in sequence recovery especially when the recovery of the base model is low. These results support that the noise in the input residue context can significantly limit sequence design performance and lead to suboptimal sequence designs. Using entropy-based masking to filter out low-quality residue predictions is an effective strategy for improving sequence recovery. The fact that the drop in sequence recovery is more pronounced when the recovery of the base model is low, suggesting that the masking operation is particularly important in cases where the input sequence information is less reliable.

In FIG. 3B, the confusion matrix of the method according to the embodiments of the subject invention performed on ESM model on CATH is calculated and shown. A darker cell indicates that a larger portion of the residues of the native type is predicted to be the corresponding type on the horizontal axis. It can be observed that the residue types that the model tends to confuse are physicochemically similar types, such as ILE vs VAL and GLU vs LYS. Mantel test has shown that the confusion matrix is highly correlated with the BLOSUM62 amino acid substation matrices (p-value 0.0001, alpha=0.05). In FIGS. 3C and 3D, the sequence recovery is broken down on CATH to different amino acid types and secondary structures (H stands for 3₁₀ helix, α helix and π helix, E for isolated beta-bridge residue and strand, and C for bend, turn and coil). Therefore, the method according to the embodiments of the subject invention demonstrates improvement in the recovery of both hydrophilic and hydrophobic residues, with slightly greater improvement seen for residues located on helices and bends, turns or coils.

Whether the designed sequences can fold into the target backbones by predicting their structure with Alphafold2 is assessed. The TM-score and root-mean-square deviation (RMSD) for the backbone heavy atoms are computed. FIGS. 3E and 3F show the results of the predicted structures of sequences designed for 2KCD and 3A57. It is observed that the sequences designed by the ESM according to the embodiments of the subject invention are predicted to fold into structures more similar to native ones than those designed by the conventional ESM, as evidenced by higher TM-scores and lower RMSD. The results suggest that the method not only improves the recovery rate of the target sequences but also generates sequences that are more structurally similar to the native ones, indicating potentials for practical protein design applications.

### Partial Sequence Design

### Evaluation on Benchmarks

TABLE 2
Median Sequence Recovery Rates and nssr
Scores on EnzBench and BR EnzBench

| | EnzBench | | BR_EnzBench | |
|---|---|---|---|---|
| | Recovery % | nssr % | Recovery % | nssr % |
| Conventional | 41.38 | 57.89 | 29.41 | 47.83 |
| GVP-GNN | | | | |
| Convention | 50.00 | 70.00 | 40.91 | 60.00 |
| ProteinMPNN-C | | | | |
| Model of the | 57.89 | 73.68 | 43.48 | 60.87 |
| subject invention | | | | |

For partial sequence design, the models are evaluated on following two benchmarks.

EnzBench: EnzBench is a standard sequence recovery benchmark comprising 51 proteins [36]. Designing methods are required to recover the native residues on protein design shells with other residues fixed. This benchmark is designed to test the method's ability to model protein binding and overall stability.

BR_EnzBench: BR_EnzBench [33] aims to test the method's ability to remodel the chosen protein structure. It randomly selects 16 proteins from EnzBench benchmark and uses the Backrub server to create an ensemble of 20 near-native conformations for each protein. To further increase the designing difficulty, all residues on the design shell are mutated to alanine, and conformations are then energy-minimized. When evaluated on EnzBench and BR_EnzBench, types of residues not on design shells are fixed and available to the models.

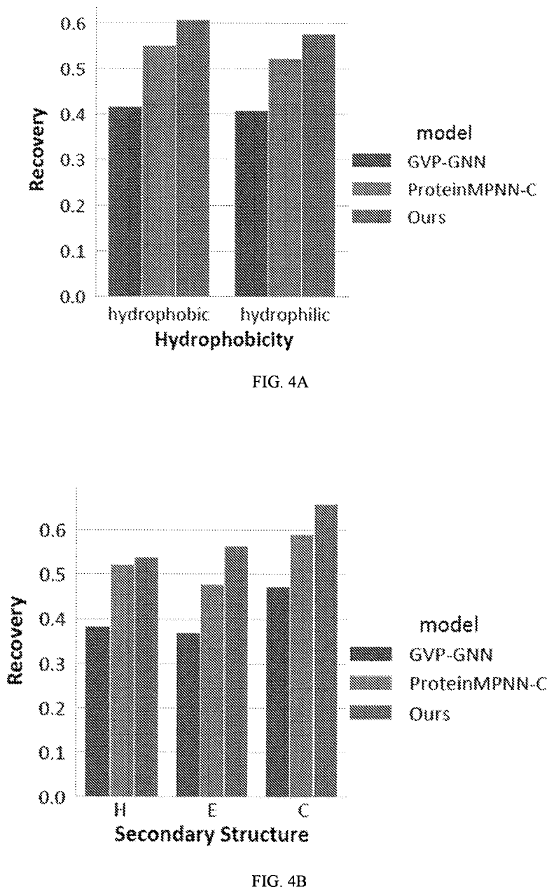

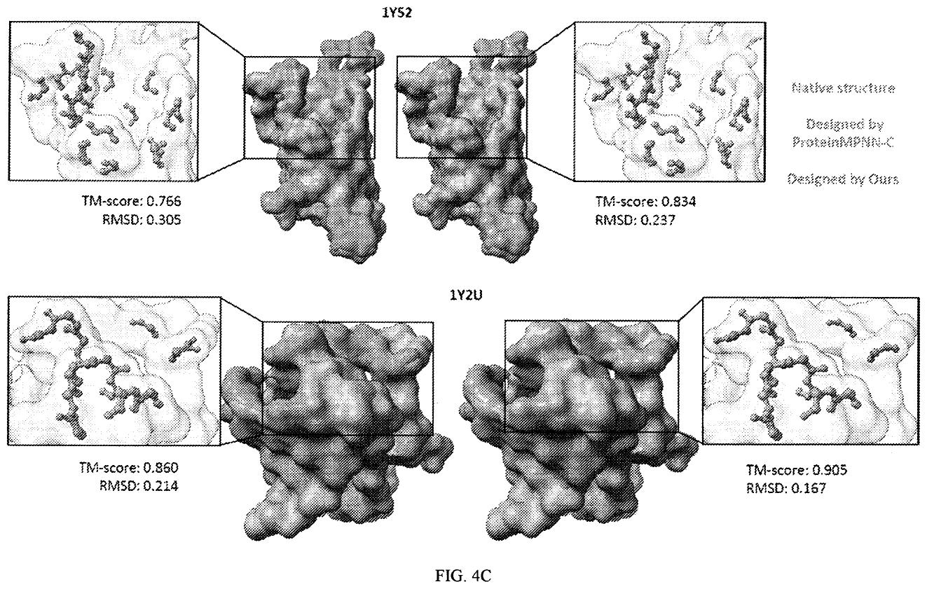

For partial sequence design, as base models are not required, the method is compared with the GVP-GNN model and the ProteinMPNN-C model, which are also trained on the same training set for fair comparison. Each model is provided with the same partial input sequence and report recovery rates and nssr scores for residues on design shells as shown in Table 2. The model according to the embodiments of the subject invention achieves the highest recovery and nssr on both benchmarks. The recovery rate on EnzBench for different amino acids and secondary structures are further analyzed as shown in FIGS. 4A-4C. The method according to the embodiments of the subject invention significantly improves the recovery of both hydrophobic and hydrophilic residues and performs better on strands and isolated beta-bridge residues. Further, Alphafold2 is employed to evaluate the designed structure's similarity and only heavy atoms on the design shell backbones are considered. FIG. 4C shows the designed structures of 1Y52 and 1Y2U, which have 19 and 13 designable residues, respectively, with design shell residues shown by atoms. The method according to the embodiments of the subject invention performs better in recovering the design shell structure, with higher TM-scores and lower RMSD.

### Application on Transposon-Associated Transposase B

Transposon-associated transposase B (TnpB) is considered to be an evolutionary precursor to CRISPR-Cas system effector protein [38]. TnpB (408 amino acids) in the Deinococcus radiodurans ISDra2 element has been demonstrated to function as a hypercompact programmable RNA-guided DNA endonuclease [39], and its miniature size is suitable for adeno-associated virus-based delivery. However, TnpB exhibits moderate gene editing activity in mammalian cells, limiting its therapeutic application.

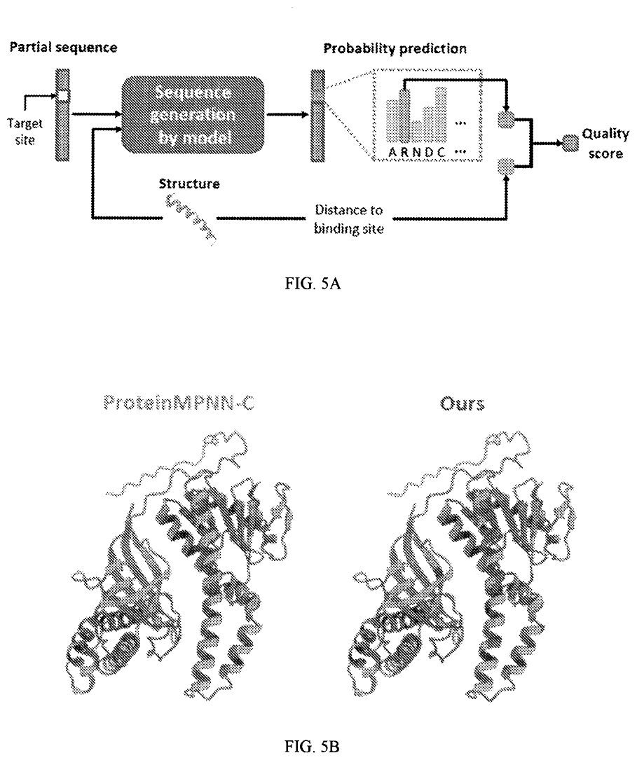

The editing activity of TnpB is improved through the design of single-point mutations. In this case, the design of a single-point mutation as a partial sequence design is provided, with only one residue being designable and all others being fixed. With the empirical intuition that a more positively charged surface may potentially improve activity, the mutation target is restricted to the most positively charged amino acid, arginine (R), and the candidate mutation sites are restricted to surface residues. The model according to the embodiments of the subject invention is configured to compute a quality score for every candidate site, as illustrated in FIG. 5A. For each site to be designed, the target site is masked in the native sequence to get the input partial sequence and the input backbone structure is the wild-type backbone predicted by Alphafold2. The model according to the embodiments of the subject invention then predicts the type of the target site in the form of a probability distribution over all amino acid types. A model that can effectively learn the residues' interaction and relationships may give a higher probability to the types that are more compatible with the given context. Hence, the predicted probability for R is taken as a measure of mutant stability. Furthermore, the distance between the Ca of the target position and the center of the predicted binding site are considered, as empirically mutation sites close to the binding site are more likely to bring forth improvements. The two scores are combined to obtain a quality score measuring how likely mutation sites can yield stable and improved mutants. All candidate sites are ranked by their quality scores and the top 20 are taken as the recommended mutation points.

The model according to the embodiments of the subject invention and ProteinMPNN-C are employed for mutant design following the above procedure. 20 mutation points recommended by two models are displayed in FIG. 5B. To test TnpB variants activity in human cells (HEK293T), plasmids encoding the TnpB variants fused with N- and C terminal nuclear localization (NLS) sequences and reRNA construct targeting a EMX1 site in human genomic DNA (gDNA) are transiently transfected into HEK293T cells. After 96 hours, gDNA is extracted and analyzed by sequencing for the presence of insertions and deletions (Indels) at the targeted cleavage sites. The experiment results indicate that 6 arginine substitutions designed by the model according to the embodiments of the subject invention achieve above 1.2-fold improvement in indel activity relative to TnpB WT and 3-fold improvement relative to the results of the ProteinMPNN-C model, as shown by FIG. 5C.

The experiment demonstrates that the model according to the embodiments of the subject invention is effective at modeling residue interactions within a structural environment and generating sequences that best fit a given 3D context. Thus, it can be employed in combination with other property measures for redesigning existing proteins and improving their stability or other qualities that depend on protein stability.

### Discussion on Model Designs

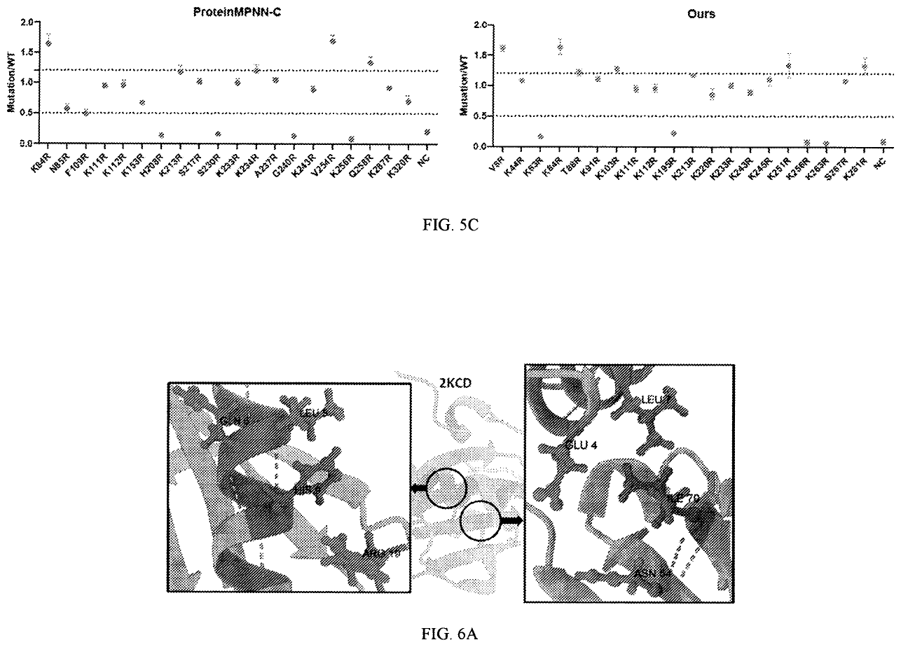

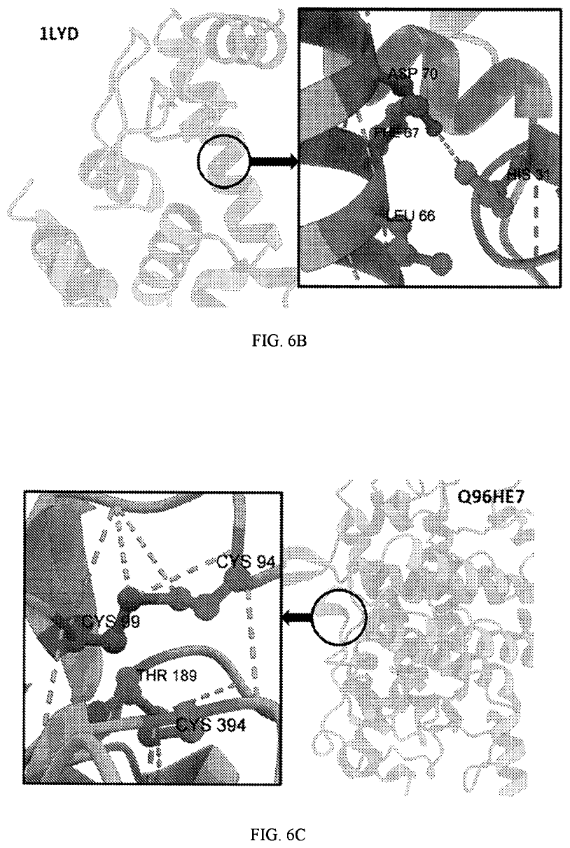

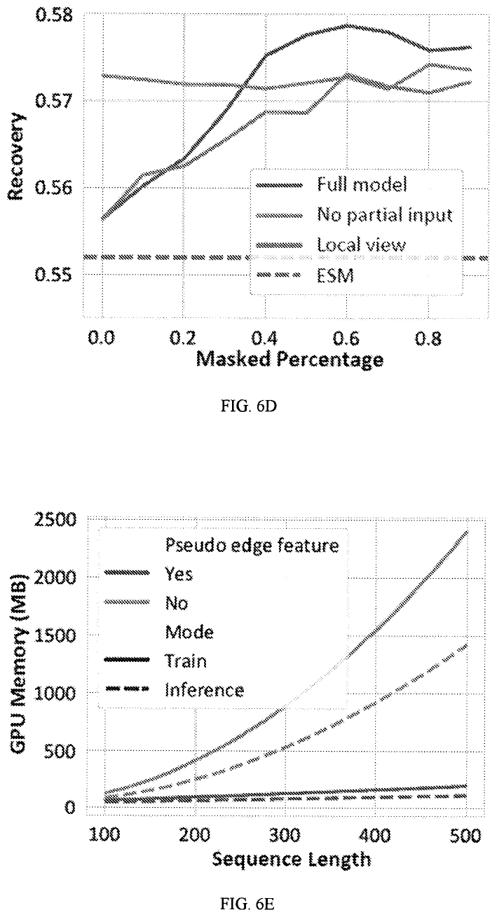

Several key designs in the model according to the embodiments of the subject invention are investigated. For the global attention layers, it is found that the layers can learn and exploit meaningful residue interactions. Specifically, the residues that a target residue most attends to, that is, the residues with the highest attention score are examined. It is observed that many important inter-residue interactions are well learned and represented by the attention operation. FIGS. 6A-6C provide examples of three chemical bonds in protein structures, where the blue residue represents the target residue, and the three residues with the highest mean attention score are shown in orange. For instance, in the case of HIS 9 on 2KCD, LEU 5 is among its most attended residues. HIS 9 forms a hydrogen bond with LEU 5 on the α helix, and the interaction is well learned by the attention layers. Similarly, ILE 70 on the sheet heavily attends to ASN 54, which it forms a hydrogen bond with. For T4-lysozyme (1LYD), ASP 70 forms a hydrogen bond with LEU 66 and a salt bridge with HIS 31, and both residues are among the most attended residues. The attention operation also captures a disulfide bond between CYS 99 and CYS 94 of human Ero1-alpha (Q96HE7).

TABLE 3
Recovery of the Model and Two Ablated Models, with
Either No Global Attention View or No Partial
Sequence Input, on EnzBench and BR EnzBench

| | | |
|---|---|---|
| Global | Partial | Recovery % |

| attention | input | EnzBench | BR_EnzBench |
|---|---|---|---|
| | ✓ | 55.56 | 42.86 |
| ✓ | | 56.52 | 35.71 |
| ✓ | ✓ | 57.89 | 43.48 |

Further validation is performed on the effectiveness of two key components of the method according to the embodiments of the subject invention: global attention method and partial sequence input method. Accordingly, two ablated models are trained: (1) a model without the global attention view, where residues only attend to their neighbors on graphs, and pseudo edge features are therefore not used; and (2) a model without partial sequence input during training, where all residue types are set to unknown. The recovery rates of these ablated models are compared with the full model on EnzBench and BR_EnzBench as shown in Table 3 above and it is found that removing either component results in a drop in the model performance. Notably, the model without partial sequence input exhibits a significantly larger performance degradation on BR_EnzBench, indicating that when input structures are not accurate, the ability to utilize sequence information becomes more important, whereas training without partial input inhibits the model from learning to encode and utilize context residues. The robustness of these models is also tested by evaluating input sequence noise in the entire sequence design. For sequences generated by the base model ESM, different percentages of residues are masked with the highest entropy, and the model's median recovery rates are plotted on the CATH benchmark in FIG. 6D. The model trained without partial sequence cannot utilize input partial sequence, and thus exhibits relatively stable performance. The recovery rates of the other two models first tend to increase when more noisy residues are removed and drops slightly when too many residues are masked and less sequence information is left. The model with a global attention view consistently outperforms the model with the local view, demonstrating a better ability to leverage the input residue context, as the residue information is available to every residue node, not just the ones close to them. Finally, the memory conserved by using the pseudo edge features is shown. As illustrated in FIG. 6E, the memory-efficient global graph attention layers run in linear memory complexity. Performing global attention without pseudo edge features increases the memory complexity to quadratic, due to the construction of fully connected graphs.

According to the embodiments of the subject invention, better modeling and learning of inter-body interactions within protein structures are studied for inverse protein folding. The two-pronged approach that incorporates a partial sequence input method and a memory-efficient global graph attention method is adopted, which work jointly to achieve effective selection and utilization of high-quality inter-residue interactions. The experiment results demonstrate that the approach is able to capture meaningful inter-residue bonds and achieves state-of-the-art sequence recovery on several protein design benchmarks. The model can be applied to redesign TnpB, resulting in the successful discovery of six mutants with enhanced editing activity. The results demonstrate great potentials for applying the model to proteins design with improved functional properties.

### Materials and Methods

### Graph Representation of Proteins

A protein structure is represented as a proximity graph G=(V,E), where V={v₁, v₂, . . . , v_{N}} denotes the residue nodes and E={eᵢⱼ} denotes the directed edges form vⱼ to vᵢ, where residue vⱼ is among the k=30 nearest neighbors of vᵢ in terms of C_{α} distance. Each node vᵢ has the following features:
sin and cos value of dihedral angles;
unit vectors from the previous and next residues on sequence to vᵢ in terms of C_{α} position.

Each edge eᵢⱼ has following features:
Gaussian radial basis functions encoding of interatomic distances between N, C_{α}, C, O and a virtual C_{β}, and encoding of distance on sequence i-j [20];
unit vector from vⱼ to vᵢ in terms of C_{α} position.

Then, two geometric vector perceptrons layers are employed to embed the extracted features.

The input partial sequence is embedded as node features. Masked residues with unknown residue type are treated as a special type “unknown”. The sequence node features are concatenated with structural node features. Resulting node features are denoted as H⁰∈R^{N×d}, where h⁰ᵢ∈R^{d} denotes the feature of vᵢ. Resulting edge features are denoted as E⁰∈R^{N×k×d}, where Eᵢ⁰∈R^{k×d} is the features of k neighbors of vᵢ and e⁰ᵢⱼ∈R^{d} denotes the feature of edge from vⱼ to vᵢ.

### Memory-Efficient Global Graph Attention Model

Attention is first introduced in Transformer model [25]. Let H∈R^{N×d} denote the d-dimension features of the input sequence with length N. The self-attention module updates the input features according to the following equations:

$$SelfAtten ( H ) = AV , \qquad ( 1 )$$

$$A = Softmax {(}^{\frac{𝒬 {K}^{T}}{\sqrt{{d}_{K}}} )} , \qquad ( 2 )$$

$$K = {HW}_{K} , \qquad ( 3 )$$

$$Q = {HW}_{Q} ,$$

$$V = {HW}_{V} ,$$

where W_{K}∈R^{d×dK}, W_{Q}∈R^{d×dK}, W_{V}∈R^{d×dV} are parameters to map H to keys, queries and values.

Transformer architecture may be employed for learning on graphs, with nodes denoted as sequence tokens. To utilize the global view provided by the original self-attention on graphs with edge features, previous works generally incorporate edge features into the attention matrix A:

$$A = Softmax {(}^{f ( \frac{𝒬 {K}^{T}}{\sqrt{{d}_{K}}} ) , ϕ ( E ) ) )} , \qquad ( 4 )$$

where E∈R^{N×N×d} is the d-dimension edge features between each pair of nodes, ϕ estimates the correlations of node pairs from edge features, which may be linear transformation [28] or more sophisticated functions [30], and f is an aggregation function, which may be element-wise addition [28, 30] or multiplication [28]. These conventional methods have two limitations though. First, to construct the edge feature matrix E, fully connected graphs are required as input and the memory complexity is determined to be O(N²). Second, the edge features are not fully leveraged. They are only involved in attention computation and cannot be used to update node features or vice versa.

The model according to the embodiments of the subject invention comprises a stack of L Memory-Efficient Global Graph Attention Layers. In each layer, nodes can globally attend to all other nodes and edge features between node pairs serve as additive attention bias terms. For non-existing edges, one solution is to convert arbitrary graphs to fully connected graphs before entering the encoder, then Equation (4) may be applied. The procedures can be done by setting k to a large enough number or using a fixed masking value/vector for non-existing edges. The operation increases memory complexity from O(N×k) to O(N²). To avoid the conversion, a learnable pseudo edge feature is adopted in each layer. Let H′ and E′ denote the input features of the lth layer and H⁰, E⁰ are inputs of the first layer. The attention is computed based on Equations (5)-(7) below:

$${A}^{l} = Softmax {(}^{\frac{{𝒬}^{l} {( {K}^{l} )}^{T}}{\sqrt{d}} + {B}^{l} )} , \qquad ( 5 )$$

$${B}_{ij}^{l} = \{ \begin{matrix}{( {w}_{B}^{l} )}^{T} {e}_{ij}^{l} & j ∈ {𝒩}_{i} \\ {( {w}_{B}^{l} )}^{T} {β}^{l} & j ∉ {𝒩}_{i}\end{matrix} \qquad ( 6 )$$

$${K}^{l} = {H}^{l} {W}_{K}^{l} , \qquad ( 7 )$$

$${𝒬}^{l} = {H}^{l} {W}_{𝒬}^{l} ,$$

where B^{l} is the attention bias, W_{K}^{l}∈R^{d×d}, W_{Q}^{l}∈R^{d×d}, w_{B}^{l}∈R^{d} are parameters, β^{l}∈R^{d} is the pseudo edge feature in layer l. Learning a pseudo edge feature for each layer is more adaptive and flexible than using one fixed masking value across all layers, providing better approximation for fully connected graphs.

The attention score A^{l} is then utilized to aggregate node features as well as the edge features. Node features are weighted and summed as in the vanilla Transformer. The edge features are aggregated with normalized weights and concatenated with aggregated node features. Finally, a linear layer is employed to map the concatenated feature to dimension d:

$${\hat{h}}_{i}^{l} = [ {\sum }_{j} {A}_{ij}^{l} {V}_{j}^{l} \vert \vert {\sum }_{j ∈ {𝒩}_{i}} \frac{{A}_{ij}^{l}}{{γ}_{i}^{l}} {e}_{ij}^{l} ] {W}_{N}^{l} , \qquad ( 8 )$$

$${V}^{l} = {H}^{l} {W}_{V}^{l} , \qquad ( 9 )$$

where W_{V}^{l}∈R^{d×d}, W_{N}^{l}∈R^{2d×d} are parameters, ∥ denotes concatenation operation, and γᵢ^{l} is a normalization term to normalize the sum of the edge weights to 1.

Next, a residue connection and layer normalization are adopted to output the final updated node features:

$${H}^{l + 1} = LayerNorm ( {H}^{\hat{} l} + {H}^{l} ) . \qquad ( 10 )$$

The edge features are then updated as follows, with W_{E}ⁱ∈R^{3d×d} being parameters:

$${e}^{\hat{}} lij = [ hli + 1 ‖ elij ‖ hlj + 1 ] WEl , \qquad ( 11 )$$

$${E}^{l + 1} = LayerNorm ( {E}^{\hat{} l} + {E}^{l} ) . \qquad ( 12 )$$

To leverage the edge features under the global attention method, compared with O(N²) by previous works, the Memory-Efficient Global Graph Attention only needs O(N×k+L) additional memory, allowing operations on longer sequences.

The output node features from the last layer are mapped to the distribution over 20 residue types through a linear layer with parameter W_{P}∈Rd×20:

$${p}_{i} = Softmax ( {h}^{{L}_{i}} {W}_{P} ) . \qquad ( 13 )$$

Negative log-likelihood loss is utilized during the training processes.
Entire Sequence Design with Base Model

During the training processes, residues are randomly sampled and their types are randomly set visible to construct an input partial sequence. During inference in entire sequence design setting, an entropy-based partial sequence construction method is employed. Suppose pᵢ^{b} is the probability distribution of vᵢ predicted by a base model, the entropy enᵢ^{b} of distribution pᵢ^{b} is computed by:

$${en}^{{b}_{i}} = E [ - log {p}^{{b}_{i}} ] . \qquad ( 14 )$$

Residues with the least entropy are selected and retained while others are masked. The partial sequence is fed into the model to get the probability predictions pᵢ with entropy enᵢ. Finally, the predictions from the base model and the model according to the embodiments of the subject invention are weighted by their entropy and fused together:

$${\hat{p}}_{i} = \frac{exp ( - {en}_{i} )}{exp ( - {en}_{i} ) + exp ( - {en}_{i}^{b} )} {p}_{i} + \frac{exp ( - {en}_{i}^{b} )}{exp ( - {en}_{i} ) + exp ( - {en}_{i}^{b} )} {p}_{i}^{b} \qquad ( 15 )$$

The final predicted residue type is the argmax of \hat{ }pᵢ.

### Experiment Details of TnpB Design

Plasmid Vector Construction: The TnpB gene is optimized for expression in human cells through codon optimization and the optimized sequence is synthesized for vector construction (Sangon Biotech, Shanghai, China). The final optimized sequence is inserted into a pST1374 vector, which contains a CMV promoter and nuclear localization signal sequences at both the 5′ and 3′ termini. The reRNA sequences are synthesized and cloned into a pGL3-U6 vector. Spacer sequences (EMX1: 5′-ctgtttctcaggatgtttgg-3′ (SEQ ID NO: 11)) are cloned into by digesting the vectors with BsaI restriction enzyme (New England BioLabs, Ipswich, MA) for 2 hours at 37° C. The resulting vector constructs are verified by Sanger sequencing to ensure accuracy.

TnpB Engineering: The construction of TnpB mutants is carried out by site-directed mutagenesis. PCR amplifications are performed using Phanta Max Super-Fidelity DNA Polymerase (Vazyme, Nanjing, Jiangsu, China). The PCR products are then ligated using 2× MultiF Seamless Assembly Mix (ABclonal, Woburn, MA). Next, ligated products are transformed into DH5α E. coli cells. The success of the mutagenesis is confirmed by Sanger sequencing. The modified plasmid vectors are purified using a TIANpure Midi Plasmid Kit (TIANGEN, Beijing, China).

Cell Culture and Transfection: HEK293T cells are maintained in Dulbecco's modified Eagle medium (Gibco, Waltham, MA) supplemented with 10% fetal bovine serum (Gemini, West Sacramento, California) and 1% penicillin-streptomycin (Gibco) in an incubator (37° C., 5% CO₂). HEK293T cells are transfected at 80% confluency with a density of approximately 1×10⁵ cells per 24-well using ExFect Transfection Reagent (Vazyme). For indel analysis, 500 ng of TnpB plasmid plus 500 ng of reRNA plasmid is transfected into 24-well cells.

DNA Extraction and Deep Sequencing: The genomic DNA of HEK293T cells is extracted using QuickExtract DNA Extraction Solution (Lucigen, Middleton, WI). Samples are incubated at 65° C. for 60 minutes and at 98° C. for 2 minutes. The lysate is used as a PCR template. The first round PCR (PCR1) is conducted with barcoded primers to amplify the genomic region of interest using Phanta Max Super-Fidelity DNA Polymerase (Vazyme). The products of PCR1 are pooled in equal moles and purified for the second round of PCR (PCR2). The PCR2 products are amplified using index primers (Vazyme) and purified by gel extraction for sequencing on the Illumina NovaSeq platform. The specific barcoded primers used in PCR1 are listed in Table 4.

TABLE 4
The Barcoded Primers Used in PCR1.

| | |
|---|---|
| EMX1- | ACACTCTTTCCCTACACGACGCTCTTCCGATCTGAGggtggttcaggectccttcccac |
| PCR- | (SEQ ID NO: 1) |
| seq-F1 | |
| EMX1- | ACACTCTTTCCCTACACGACGCTCTTCCGATCTTAAggtggttcaggcctccttcccac |
| PCR- | (SEQ ID NO: 2) |
| seq-F2 | |
| EMX1- | ACACTCTTTCCCTACACGACGCTCTTCCGATCTTCAggtggttcaggcctecttcccac |
| PCR- | (SEQ ID NO: 3) |
| seq-F3 | |
| EMX1- | ACACTCTTTCCCTACACGACGCTCTTCCGATCTTCGggtggttcaggcctccttcccac |
| PCR- | (SEQ ID NO: 4) |
| seq-F4 | |
| EMX1- | ACACTCTTTCCCTACACGACGCTCTTCCGATCTTGCggtggttcaggcctccttcccac |
| PCR- | (SEQ ID NO: 5) |
| seq-F5 | |
| EMX1- | GTGACTGGAGTTCAGACGTGTGCTCTTCCGATCTACCcaagatgctaagtgatgacagg |
| PCR- | (SEQ ID NO: 6) |
| seq-R1 | |
| EMX1- | GTGACTGGAGTTCAGACGTGTGCTCTTCCGATCTCAGcaagatgctaagtgatgacagg |
| PCR- | (SEQ ID NO: 7) |
| seq-R2 | |
| EMX1- | GTGACTGGAGTTCAGACGTGTGCTCTTCCGATCTCCTcaagatgctaagtgatgacagg |
| PCR- | (SEQ ID NO: 8) |
| seq-R3 | |
| EMX1- | GTGACTGGAGTTCAGACGTGTGCTCTTCCGATCTCTAcaagatgctaagtgatgacagg |
| PCR- | (SEQ ID NO: 9) |
| seq-R4 | |
| EMX1- | GTGACTGGAGTTCAGACGTGTGCTCTTCCGATCTGTAcaagatgctaagtgatgacagg |
| PCR- | (SEQ ID NO: 10) |
| seq-R5 | |

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

[1] Gao W, Mahajan S P, Sulam J, Gray J J. Deep learning in protein structural modeling and design. Patterns. 2020; p. 100142.
[2] Huang P S, Feldmeier K, Parmeggiani F, Fernandez Velasco D A, H\ddot{ }ocker B, Baker D. De novo design of a four-fold symmetric TIM-barrel protein with atomic-level accuracy. Nature chemical biology. 2016; 12(1): 29-34.
[3] Lin Y R, Koga N, Tatsumi-Koga R, Liu G, Clouser A F, Montelione G T, et al. Control over overall shape and size in de novo designed proteins. Proceedings of the National Academy of Sciences. 2015; 112(40): E5478-E5485.
[4] Anand-Achim N, Eguchi R R, Mathews I I, Perez C P, Derry A, Altman R B, et al. Protein sequence design with a learned potential. bioRxiv. 2021; p. 2020-01.
[5] Alford R F, Leaver-Fay A, Jeliazkov J R, O'Meara M J, DiMaio F P, Park H, et al. The Rosetta all-atom energy function for macromolecular modeling and design. Journal of chemical theory and computation. 2017; 13(6): 3031-3048.
[6] Slovic A M, Summa C M, Lear J D, DeGrado W F. Computational design of a water-soluble analog of phospholamban. Protein Science. 2003; 12(2): 337-348.
[7] Jiang L, Althoff E A, Clemente F R, Doyle L, R\ddot{ }othlisberger D, Zanghellini A, et al. De novo computational design of retro-aldol enzymes. science. 2008; 319(5868): 1387-1391.
[8] Pabo C. Molecular technology: designing proteins and peptides. Nature. 1983; 301(5897): 200-200.
[9] Wilmanns M, Eisenberg D. Three-dimensional profiles from residue pair preferences: identification of sequences with beta/alpha-barrel fold. Proceedings of the National Academy of Sciences. 1993; 90(4): 1379-1383.
[10] Zhou X, Xiong P, Wang M, Ma R, Zhang J, Chen Q, et al. Proteins of well-defined structures can be designed without backbone readjustment by a statistical model. Journal of structural biology. 2016; 196(3): 350-357.
[11] Rohl C A, Strauss C E, Misura K M, Baker D. Protein structure prediction using Rosetta. In: Methods in enzymology. vol. 383. Elsevier; 2004. p. 66-93.
[12] Du Y, Meier J, Ma J, Fergus R, Rives A. Energy-based models for atomicresolution protein conformations. arXiv preprint arXiv: 200413167. 2020.
[13] Jumper J, Evans R, Pritzel A, Green T, Figurnov M, Ronneberger O, et al. Highly accurate protein structure prediction with AlphaFold. Nature. 2021; 596(7873): 583-589.
[14] Norn C, Wicky B I, Juergens D, Liu S, Kim D, Koepnick B, et al. Protein sequence design by explicit energy landscape optimization. bioRxiv. 2020.
[15] Zhang Y, Chen Y, Wang C, Lo C C, Liu X, Wu W, et al. ProDCoNN: Protein design using a convolutional neural network. Proteins: Structure, Function, and Bioinformatics. 2020; 88(7): 819-829.
[16] Li Z, Yang Y, Faraggi E, Zhan J, Zhou Y. Direct prediction of profiles of sequences compatible with a protein structure by neural networks with fragment-based local and energy-based nonlocal profiles. Proteins: Structure, Function, and Bioinformatics. 2014; 82(10): 2565-2573.
[17] O'Connell J, Li Z, Hanson J, Heffernan R, Lyons J, Paliwal K, et al. SPIN2: Predicting sequence profiles from protein structures using deep neural networks. Proteins: Structure, Function, and Bioinformatics. 2018; 86(6): 629-633.
[18] Ingraham J, Garg V, Barzilay R, Jaakkola T. Generative models for graph-based protein design. Advances in neural information processing systems. 2019; 32.
[19] Jing B, Eismann S, Suriana P, Townshend R J, Dror R. Learning from protein structure with geometric vector perceptrons. arXiv preprint arXiv: 200901411. 2020.
[20] Dauparas J, Anishchenko I, Bennett N, Bai H, Ragotte R J, Milles L F, et al. Robust deep learning based protein sequence design using ProteinMPNN. bioRxiv. 2022.
[21] Hsu C, Verkuil R, Liu J, Lin Z, Hie B, Sercu T, et al. Learning inverse folding from millions of predicted structures. bioRxiv. 2022.
[22] Liu Y, Zhang L, Wang W, Zhu M, Wang C, Li F, et al. Rotamer-free protein sequence design based on deep learning and self-consistency. Nature Computational Science. 2022 July; 2(7): 451-462.
[23] Li B, Tian J, Zhang Z, Feng H, Li X. Multitask non-autoregressive model for human motion prediction. IEEE Transactions on Image Processing. 2020; 30:2562-2574.
[24] Huang R, Hu H, Wu W, Sawada K, Zhang M. Dance Revolution: Long Sequence Dance Generation with Music via Curriculum Learning. CoRR. 2020; abs/2006.06119.
[25] Vaswani A, Shazeer N, Parmar N, Uszkoreit J, Jones L, Gomez A N, et al. Attention is all you need. In: Advances in neural information processing systems; 2017. p. 5998-6008.
[26] Dwivedi V P, Bresson X. A Generalization of Transformer Networks to Graphs. CoRR. 2020; abs/2012.09699.
[27] Hu Z, Dong Y, Wang K, Sun Y. Heterogeneous Graph Transformer. In: Huang Y, King I, Liu T, van Steen M, editors. WWW '20: The Web Conference 2020, Taipei, Taiwan, Apr. 20-24, 2020. ACM/IW3C2; 2020. p. 2704-2710.
[28] Hussain M S, Zaki M J, Subramanian D. Edge-augmented Graph Transformers: Global Self-attention is Enough for Graphs. CoRR. 2021; abs/2108.03348.
[29] Bergen L, O'Donnell T J, Bahdanau D. Systematic Generalization with Edge Transformers. In: Ranzato M, Beygelzimer A, Dauphin Y N, Liang P, Vaughan J W, editors. Advances in Neural Information Processing Systems 34: Annual Conference on Neural Information Processing Systems 2021, NeurIPS 2021 Dec. 6-14, 2021, virtual; 2021. p. 1390-1402.
[30] Ying C, Cai T, Luo S, Zheng S, Ke G, He D, et al. Do Transformers Really Perform Bad for Graph Representation? CoRR. 2021; abs/2106.05234.
[31] Wang J, Cao H, Zhang J Z, Qi Y. Computational protein design with deep learning neural networks. Scientific reports. 2018; 8(1): 1-9.
[32] Qi Y, Zhang J Z. DenseCPD: improving the accuracy of neural-networkbased computational protein sequence design with DenseNet. Journal of chemical information and modeling. 2020; 60(3): 1245-1252.
[33] L\ddot{ }offler P, Schmitz S, Hupfeld E, Sterner R, Merkl R. Rosetta: MSF: a modular framework for multi-state computational protein design. PLOS computational biology. 2017; 13(6): e1005600.
[34] Henikoff S, Henikoff J G. Amino acid substitution matrices from protein blocks. Proceedings of the National Academy of Sciences. 1992; 89(22): 10915-10919.
[35] Mantel N. The detection of disease clustering and a generalized regression approach. Cancer research. 1967; 27(2 Part 1): 209-220.
[36] Niv\acute{ }on L G, Bjelic S, King C, Baker D. Automating human intuition for protein design. Proteins: Structure, Function, and Bioinformatics. 2014; 82(5): 858-866.
[37] Lauck F, Smith C A, Friedland G F, Humphris E L, Kortemme T. RosettaBackrub—a web server for flexible backbone protein structure modeling and design. Nucleic acids research. 2010; 38 (suppl 2): W569-W575.
[38] Makarova K S, Wolf Y I, Iranzo J, Shmakov S A, Alkhnbashi O S, Brouns S J, et al. Evolutionary classification of CRISPR-Cas systems: a burst of class 2 and derived variants. Nature Reviews Microbiology. 2020; 18(2): 67-83.
[39] Karvelis T, Druteika G, Bigelyte G, Budre K, Zedaveinyte R, Silanskas A, et al. Transposon-associated TnpB is a programmable RNA-guided DNA endonuclease. Nature. 2021; 599(7886): 692-696.
[40] Yuan Q, Chen S, Rao J, Zheng S, Zhao H, Yang Y. AlphaFold2-aware protein-DNA binding site prediction using graph transformer. Briefings in Bioinformatics. 2022; 23(2): bbab564.

We claim:

1. A machine-learning based method for protein sequence design, comprising:
receiving information of residues of a protein sequence;
determining if any residue of the protein sequence is known;
performing entire sequence design if it is determined that no residue of the protein sequence is known;
performing partial sequence design if it is determined that at least one residue of the protein sequence is known; and
generating an entire sequence non-iteratively.

2. The machine-learning based method of claim 1, wherein the performing entire sequence design comprises performing an entropy-based prediction-selection method in combination with a base model to remove noise in input residue context.

3. The machine-learning based method of claim 2, wherein the performing an entropy-based prediction-selection method comprises computing an entropy of predicted distributions at each position, retaining residues having entropies lower than or equal to a threshold value, and masking other residues having entropies greater than the threshold value.

4. The machine-learning based method of claim 2, wherein the base model is a GVP-GNN model.

5. The machine-learning based method of claim 2, wherein the base model is a ProteinMPNN model.

6. The machine-learning based method of claim 2, wherein the base model is a ProteinMPNN-C model.

7. The machine-learning based method of claim 2, wherein the base model is an ESM model.

8. A machine-learning based method for protein sequence design, comprising:
generating a portion of a sequence and removing noise in input residue context;
encoding and processing the portion of the sequence and backbone structure to obtain graph features;
performing memory-efficient global graph attention layers to propagate the graph features and learn global residue interactions; and
generating an entire sequence non-iteratively.

9. The machine-learning based method of claim 8, wherein the performing memory-efficient global graph attention layers comprises enabling each residue node to learn residue interactions and gather information from the entire sequence while maintaining memory efficiency.

10. The machine-learning based method of claim 8, wherein the performing memory-efficient global graph attention layers comprises constructing a K-nearest neighbor graph from the backbone structure and node features contain dihedral angles, forward and backward vectors.

11. The machine-learning based method of claim 8, wherein edge features of the memory-efficient global graph attention layers include interatomic distances and direction vectors.

12. The machine-learning based method of claim 11, wherein the edge features are encoded by GVP layers to obtain structural embeddings.

13. The machine-learning based method of claim 11, wherein in each layer of the memory-efficient global graph attention layers, every residue node globally attends to other residues.

14. The machine-learning based method of claim 13, wherein an attention score is calculated from both the node and the edge features to determine amount of information that a target node gathers from another node.

15. The machine-learning based method of claim 14, wherein for node pairs that are not directly connected by an edge, a learnable pseudo edge feature is configured for attention calculation.

16. The machine-learning based method of claim 15, wherein each layer learns a separate pseudo edge feature that is shared by all non-existing edges.

17. The machine-learning based method of claim 16, wherein the attention score is then used to weight and sum up the node and the edge features, generating updated node features.

18. The machine-learning based method of claim 17, wherein the edge features are updated by the updated node features.

19. The machine-learning based method of claim 18, wherein the generating an entire sequence non-iteratively comprises generating the entire sequence from the node features from the last layer non-iteratively.

20. A computer program product, comprising:
a non-transitory computer-executable storage device having computer readable program instructions embodied thereon that when executed by a computer cause the computer to perform machine-learning based method for protein sequence design, the computer-executable program instruction comprising:
generating a portion of a sequence and removing noise in input residue context;
encoding and processing the portion of the sequence and backbone structure to obtain graph features;
performing memory-efficient global graph attention layers to propagate the graph features and learn global residue interactions; and
generating an entire sequence non-iteratively.


SEQUENCE LISTING (11 sequences)

SEQ ID NO: 1 - DNA, 59 bp, synthetic construct
  source 1..59: mol_type = other DNA
acactctttccctacacgacgctcttccgatctgagggtggttcaggcctccttcccac

SEQ ID NO: 2 - DNA, 59 bp, synthetic construct
  source 1..59: mol_type = other DNA
acactctttccctacacgacgctcttccgatcttaaggtggttcaggcctccttcccac

SEQ ID NO: 3 - DNA, 59 bp, synthetic construct
  source 1..59: mol_type = other DNA
acactctttccctacacgacgctcttccgatcttcaggtggttcaggcctccttcccac

SEQ ID NO: 4 - DNA, 59 bp, synthetic construct
  source 1..59: mol_type = other DNA
acactctttccctacacgacgctcttccgatcttcgggtggttcaggcctccttcccac

SEQ ID NO: 5 - DNA, 59 bp, synthetic construct
  source 1..59: mol_type = other DNA
acactctttccctacacgacgctcttccgatcttgcggtggttcaggcctccttcccac

SEQ ID NO: 6 - DNA, 59 bp, synthetic construct
  source 1..59: mol_type = other DNA
gtgactggagttcagacgtgtgctcttccgatctacccaagatgctaagtgatgacagg

SEQ ID NO: 7 - DNA, 59 bp, synthetic construct
  source 1..59: mol_type = other DNA
gtgactggagttcagacgtgtgctcttccgatctcagcaagatgctaagtgatgacagg

SEQ ID NO: 8 - DNA, 59 bp, synthetic construct
  source 1..59: mol_type = other DNA
gtgactggagttcagacgtgtgctcttccgatctcctcaagatgctaagtgatgacagg

SEQ ID NO: 9 - DNA, 59 bp, synthetic construct
  source 1..59: mol_type = other DNA
gtgactggagttcagacgtgtgctcttccgatctctacaagatgctaagtgatgacagg

SEQ ID NO: 10 - DNA, 59 bp, synthetic construct
  source 1..59: mol_type = other DNA
gtgactggagttcagacgtgtgctcttccgatctgtacaagatgctaagtgatgacagg

SEQ ID NO: 11 - DNA, 20 bp, synthetic construct
  source 1..20: mol_type = other DNA
ctgtttctcaggatgtttgg